(19) United States
(12) Patent Application Publication
(10) Pub. No.: US 2023/0141222 A1
(43) Pub. Date: May 11, 2023

(54) A method for measuring the quantity of super absorbent polymers in post-consumer absorbent sanitary products

(71) Applicant: Fater S.p.A., Spoltore (Pescara) (IT)

(72) Inventors: Tonino CARUSO, Spoltore (Pescara) (IT); Patrizia IANNECE, Spoltore (Pescara) (IT); Nicola D'ALESSIO, Spoltore (Pescara) (IT); Melania MIGLIACCIO, Spoltore (Pescara) (IT)

(21) Appl. No.: 17/912,886

(22) PCT Filed: Feb. 17, 2021
(86) PCT No.: PCT/IB2021/051321

§ 371 Date: Sep. 20, 2022

(30) Foreign Application Priority Data
Mar. 20, 2020 (IT) 102020000005995

(51) Int. Cl. C08L 1/02; G01N 33/44; C08L 33/08

(52) U.S. Cl. CPC C08L 1/02; C08L 33/08; G01N 33/442

(57) ABSTRACT

A method is provided for measuring a quantity of super absorbent polymers (SAP) in a sample obtained from post-consumer absorbent sanitary products comprising at least one portion of a portion of cellulose and/or a portion of plastic in addition to a portion of SAP, said post-consumer absorbent sanitary products having been, preferably, previously subjected to at least one treatment comprising the separation of said portions; the SAP contained in said sample comprise linear polyacrylate polymers (LPA) and/or cross-linked polyacrylate polymers (CLPA).

### FIELD OF THE INVENTION

The present description refers to methods for measuring the quantity of super absorbent polymers (SAP) in samples derived from post-consumer absorbent sanitary products that have been subjected to treatments, which include, for example, steps for sterilizing and separating into their components.

### BACKGROUND OF THE INVENTION

Absorbent sanitary products are generally composed of different materials, including, for example, plastic film, cellulose fluff, and superabsorbent polymers (SAP). These sanitary products, therefore, contain fractions of precious materials whose recovery for reuse on the market is a decidedly desirable objective; the value and intended use for convenient recycling of the different fractions largely depends on the components recovered.

The most widely used and commercially available super absorbent polymers, SAP, are generally prepared by copolymerization of one or more monomers that give rise to the basic structure (backbone) of the SAP, a basic structure that comprises a linear polyacrylate (LPA) with a crosslinker of the bifunctional type. The result is a copolymer based on cross-linked linear polyacrylate (CLPA), which has a degree of crosslinking depending on the quantity of crosslinking compound used. The polymeric network that is formed has negatively charged carboxylate groups (—COO⁻) , which, due to electrostatic repulsions, can expand thus providing spaces inside that can absorb (and retain) more or less large volumes of water or aqueous solutions (hence the definition “absorbent gelling material”, AGM). Crosslinking is also essential in order to render the copolymer insoluble in an aqueous environment.

Methods known — to date — for treating absorbent sanitary products that include the sterilization and separation of the different portions (or fractions) - or rather, the cellulose portion, the plastic portion, the SAP portion - may comprise steps wherein the post-consumer sanitary products (for example used diapers) are subjected to treatments with oxidizing compounds, for example, hydrogen peroxide, persulfate, peroxy-monosulfate and/or ozone, to achieve the chemical decontamination of these post-consumer sanitary products. However, treatments with oxidizing compounds may cause the destructuring of cross-linked super absorbent polymers (CLPA) and, consequently, the release of polymers of linear polyacrylate (LPA) in the alkaline form, essentially sodium or potassium.

In this precise context, the quantitative determination of the SAP content in post-consumer absorbent sanitary products may be altered by the poor chemical inertness of the treated material, both due to the loss of crosslinking and release of LPA, and because the saline content of the post-consumer products may alter the absorbent capacity of the polymers themselves. Consequently, methods for quantifying the residual post-consumer SAP content that are based only on the absorption capacity of the SAP may be insensitive and not reproducible.

### OBJECT AND SUMMARY OF THE INVENTION

The present description aims to provide a sensitive and reproducible method for measuring the quantity of super absorbent polymers (SAP) in cellulose, plastic, and SAP samples, wherein these samples are derived from post-consumer absorbent sanitary products which have preferably been subjected to treatments that include, for example, the sterilization and separation of the various components of cellulose, plastic, and SAP.

According to the present description, this object is achieved thanks to a method having the characteristics forming the subject of the attached claims. The claims form an integral part of the disclosure provided here in relation to the described method.

The present description provides a method for measuring the quantity of super absorbent polymers (SAP) in a sample obtained from post-consumer absorbent sanitary products comprising at least one portion of a cellulose portion and/or a plastic portion in addition to a portion of SAP and, preferably, previously subjected to at least one treatment that preferably comprises the sterilization and separation of said portions, wherein the SAP contained in said sample comprise polymers of linear polyacrylate (LPA) and/or polymers of cross-linked polyacrylate (CLPA), the method comprising the steps of:
a) contacting a first fraction of the sample with an aqueous solution comprising a known quantity C₀ of a water-soluble cation X^{Y} and obtaining a suspension comprising a solid fraction and a liquid fraction,
b) after a period of time T₁, separating the solid fraction from the liquid fraction and measuring the residual quantity C₁ of said water-soluble cation X^{Y} in said liquid fraction,
c) calculating the difference C₀-C₁ between the known quantity C₀ and the quantity C₁ of the cation X^{Y} remaining after the time period T₁,
d) contacting a second fraction of the sample with a solution comprising a salt, for a period of time T₂, and obtaining a suspension comprising a solid fraction and a liquid fraction, wherein said salt promotes the passage of said water-soluble cation X^{Y} of metabolic origin and not already bound to the SAP into said liquid fraction,
e) separating the solid fraction from the liquid fraction and contacting, for a period of time T₃, the solid fraction with a solution comprising an acid for displacing said water-soluble cation X^{Y}, of metabolic origin, already bound to the SAP and promoting its passage into said solution,
f) measuring the quantity C_{B} of said water-soluble cation X^{Y} contained in said liquid fraction obtained in step d) after the period of time T₂, and the quantity C_{H} of said water-soluble cation X^{Y} contained in said aqueous solution containing an acid, preferably HCl, after the time period T₃,
g) calculating the difference C_{H} - C_{B} this difference being indicative of the quantity of said cation contained in the sample and derived from the metabolism and bound to the SAP,
h) calculating the quantity of SAP contained in said sample as a function of the difference C₀-C₁ and of the difference C_{H}-C_{B}.

In one or more embodiments, said calculating the quantity of SAP comprises applying a proportionality coefficient z indicative of the molecular weight of the SAP monomeric unit.

In one or more embodiments, said calculating the amount of SAP further comprises applying a correction coefficient A indicative of the crosslinking degree of the SAP.

In one or more embodiments, the quantity of SAP can be calculated according to the equation E₁: A · [(C₀ - C₁) + (C_{H} - C_{B})] · z.

When the monomer unit of the SAP is C₃H₃NaO₂, the coefficient z is equal to 94.04 g/mol. The correction coefficient A is equal to 1.22 and is calculated as described below. This coefficient is independent from the cation used, from its counter-ion, as it is related to the degree of crosslinking.

The method may, furthermore, comprise the steps of:
i) contacting a third fraction of the sample to be subjected to measurement with an aqueous solution containing a salt, preferably NaCl, and obtaining a suspension comprising a solid fraction and a liquid fraction in order to obtain the solubilization and passage of the LPA fraction of the SAP into liquid phase,
l) obtaining a liquid fraction comprising LPA and a solid fraction comprising CLPA,
m) separating the liquid fraction containing LPA from the solid fraction containing CLPA,
n) contacting the solid fraction containing CLPA with an aqueous solution containing a known quantity C₀ of said cation X^{Y} for a period of time T₄, and measuring the quantity C₃ of said cation after the time period T₄ in order to obtain the quantity of CLPA in the sample to be tested,
o) adding a known quantity C₀ of said cation X^{Y} to the liquid fraction containing LPA, and measuring the quantity C₂ of the cation after the time period T₅ in order to obtain the quantity of LPA in the sample to be tested,
p) calculating the quantity of CLPA as a function of C₀ - C₃ and C_{H} and/or
q) calculating the quantity of LPA as a function of C₀ - C₂ and C_{B}.

In one or more embodiments, the quantity of CLPA is calculated according to the equation E₁: A · [(C₀ --C₃) + C_{H}] · z and/or said quantity of LPA is calculated according to the equation E₃: A · [(C₀ - C₂) - C_{B}] • z.

When the monomer unit of the SAP is C₃H₃NaO₂, the coefficient z is equal to 94.04 g/mol. The correction coefficient A is equal to 1.22 and is calculated as described below. This coefficient is independent from the cation used, from its counter-ion as it is related to the degree of crosslinking.

The reactions of CLPA and LPA with calcium ion lead to the formation of insoluble derivatives, which subtract calcium from the starting solution, whose analytical concentration C₀ is known. The “missing” calcium may, therefore, be traced back to the total quantity of SAP with the possibility of distinguishing the quantity of CLPA and LPA.

### BRIEF DESCRIPTION OF THE FIGURES

The method will now be described in detail with reference to the attached drawings, given purely by way of non-limiting example, wherein:

FIG. 1 represents the main components of the SAP and their three-dimensional structure (extracted from: BASF Superabsorbents, SAP website: www.superabsorbents.basf.com/home);

FIG. 2 represents the molecular mechanism underlying the swelling of SAP when placed in aqueous solution (extracted from: MJ Zohuriaan-Mehr, K. Kabiri, Superabsorbent Polymer Materials: A Review, Iranian Polymer Journal 17(6), 2008, 451-477);

FIG. 3 regards the titration of 1.00 g of virgin SAP-Na/K with calcium ions in 500.0 ml of water. The concentration of ions in solution is zero up to the equivalence of the precipitation reaction of the calcium SAP, after which it increases linearly;

FIG. 4 refers to the determination of the correction coefficient A, indicative of the degree of crosslinking, by means of separate titrations of 11 aliquots of 1.00 g of virgin SAP-Na/K deriving from 11 distinct commercial batches. The titrations were carried out with divalent ions deriving either from chloride salts, or from nitrate, or from sulfate, dissolved in 500.0 ml of water. The value of A was found to be independent of the nature of the cation and that of its counter-ion. The average value is equal to 1.22 ± 0.02.

### DETAILED DESCRIPTION

In the following description, numerous specific details are provided to allow a thorough understanding of embodiments. The embodiments can be put into practice without one or more of the specific details or with other methods, components, materials etc. In other cases, well-known structures, materials or operations are not shown or described in detail to avoid confusing aspects of the embodiments.

Reference throughout the present disclosure to “one embodiment” or “an embodiment” indicates that a particular aspect, structure or characteristic described with reference to the embodiment is included in at least one embodiment. Thus, forms of the expressions “in one embodiment” or “in an embodiment” at various points throughout the present description are not necessarily all referring to the same embodiment. Moreover, the particular aspects, structures or characteristics can be combined in any convenient way in one or more embodiments. The titles provided in this description are for convenience only and do not interpret the scope or object of the embodiments.

The expression “absorbent sanitary products” generally refers to disposable absorbent products, such as diapers for babies, incontinence pads for adults, sanitary towels, bed linings, etc. These absorbent products can comprise plastic, super-absorbent polymers, cellulose or even only plastic and super-absorbent polymers.

As anticipated in the preceding sections, methods are known to date for treating post-consumer absorbent sanitary products in order to obtain the separation of the various components, such as, for example, cellulose, plastic, super-absorbent polymers (SAP). A method known to date for treating and separating the different components (or fractions or portions) of post-consumer absorbent sanitary products may comprise the steps described, for example, in the document WO 2018/060827 by the same Applicant. This method may comprise the step of sterilizing the post-consumer absorbent sanitary products, of shredding the sterilized products, drying the sterilized and shredded products, separating the sterilized, shredded and dried products into the plastic, cellulose, and SAP portions.

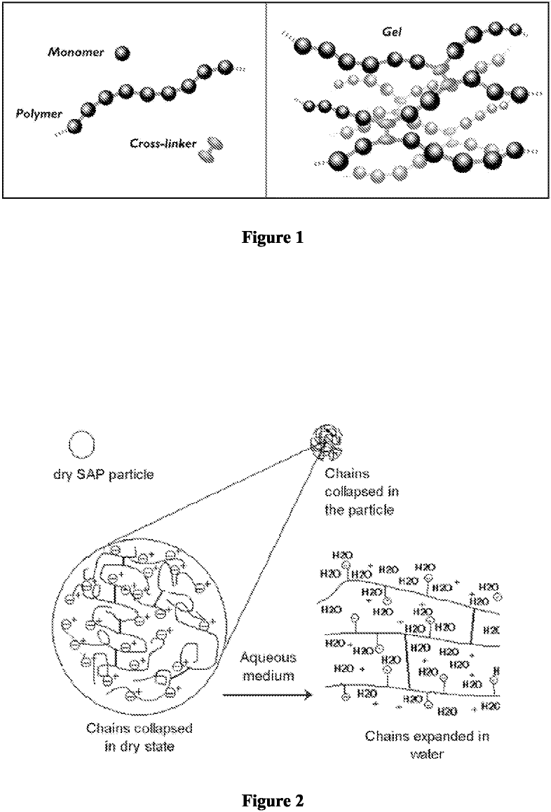

SAP are generally prepared by the copolymerization of one or more monomers (acrylic acid, sodium or potassium acrylate, and/or acrylamide; Liu M., Guo T. 2001. Preparation and swelling properties of crosslinked sodium polyacrylate. Journal of Applied Polymer Science 82: 1515-1520). As schematized in FIG. 1, these monomers produce the basic structure (backbone) of the material, or rather, a linear polyacrylate (LPA) with a bifunctional crosslinker (generally N, N-methylene-bis-acrylamide (MBA), Ethylene glycol dimethacrylate (EGDMA), Diallyl phthalate (DP), Triethylene glycol dimethacrylate (TEGDMA)). The result is a copolymer based on crosslinked linear polyacrylate (CLPA), which has a degree of crosslinking depending on the quantity of crosslinking compound used. As illustrated in FIG. 2, the polymeric network that is formed has negatively charged carboxylate groups (—COO⁻), which, due to electrostatic repulsions, can expand thus providing spaces inside the lattice that can absorb (and retain) more or less large volumes of water or aqueous solutions (hence the definition “absorbent gelling material”, AGM). Crosslinking is also essential in order to render the copolymer insoluble in an aqueous environment.

Methods known to date for treating absorbent sanitary products that include the sterilization and separation of the different fractions — or rather, the cellulose fraction, the plastic fraction, the SAP fraction — may comprise steps wherein the post-consumer sanitary products (for example, used diapers), as well as being sterilized, are subjected to treatments with oxidizing compounds, for example, hydrogen peroxide, persulfate, peroxy-monosulfate and/or ozone, to achieve, for example, decontamination from chemical compounds also derived from human metabolism However, these treatments may cause the destructuring of cross-linked super absorbent polymers (CLPA) and, consequently, the release of soluble polymers of linear polyacrylate (LPA) in the alkaline form, essentially sodium or potassium.

In this precise context, determination of the quantity of SAP can be altered by the poor chemical inertness of the treated material, both due to the loss of crosslinking between polymers and the release of LPA, and because the saline content may alter the absorbent capacity of the polymers themselves. Therefore, methods of determining the quantity of residual post-consumer SAP that use just the property of absorbing water, (W_{abs} Determination of absorbent power, NF V19-002-1993-02-French standard) are insensitive to the detection of products derived from SAP, in particular of LPA, which does not absorb. Furthermore, these methods are unreliable as they do not consider the effect of the ionic strength on the ability to absorb water itself (W_{abs}).

Moreover, these methods have been proven to be imprecise and not very reproducible as they constrain a correlation obtained by calibration on ideal samples to the relative quantitative analysis on real samples. It should be taken into account, however, that blends of virgin material used for determining the correlation value between the absorption capacity (W_{abs}) and the percentage content (%) of SAP, have characteristics that make them different from mixtures derived from post-consumer material that has been contaminated. For example, post-consumer sanitary products may comprise salts and ionic compounds (not present in a virgin material) and which are capable of significantly altering the absorbent capacity of SAP. Furthermore, these methods do not take into consideration that the absorbent capacity of the SAP may also vary as a function of the intrinsic salinity of the sample derived from the post-consumer absorbent material. These methods, therefore, although reliable and reproducible on samples of virgin material, have a poor reproducibility, on samples derived from treated post-consumer material which have a high electrolyte content, and thus retain less liquids and swell less.

The present description therefore provides a method for measuring the quantity of super absorbent polymers (SAP) in a sample obtained from post-consumer absorbent sanitary products comprising at least one portion of a cellulose portion and/or a plastic fraction in addition to a fraction of SAP, said post-consumer absorbent sanitary products, preferably, having been previously subjected to at least one treatment that comprises the sterilization and separation of said portions, wherein the SAP contained in said sample comprise polymers of linear polyacrylate (LPA) and/or polymers of cross-linked polyacrylate (CLPA), the method comprising the steps of:
a) contacting a first fraction of the sample with an aqueous solution comprising a known quantity C₀ of a water-soluble cation X^{Y} and obtaining a suspension comprising a solid fraction and a liquid fraction,
b) after a period of time T₁, separating the solid fraction from the liquid fraction and measuring the residual quantity C₁ of said water-soluble cation X^{Y} in said liquid fraction,
c) calculating the difference C₀-C₁ between the known quantity C₀ and the quantity C₁ of the cation X^{Y} remaining after the time period T₁,
d) contacting a second fraction of the sample with a solution comprising a salt, for a period of time T₂, and obtaining a suspension comprising a solid fraction and a liquid fraction, wherein said salt promotes the passage of said water-soluble cation X^{Y} of metabolic origin and not already bound to the SAP into said liquid fraction,
e) separating the solid fraction from the liquid fraction and contacting, for a period of time T₃, the solid fraction with a solution comprising an acid for displacing said water-soluble cation X^{Y} of metabolic origin already bound to the SAP and promoting its passage into said solution,
f) measuring the quantity C_{B} of said water-soluble cation X^{Y} contained in said liquid fraction obtained in step d) after the period of time T₂, and the quantity C_{H} of said water-soluble cation X^{Y} contained in said aqueous solution containing an acid, preferably HCl, after the time period T₃,
g) calculating the difference C_{H} - C_{B}, this difference being indicative of the quantity of said cation contained in the sample and derived from the metabolism and bound to the SAP,
h) calculating the quantity of SAP contained in said sample as a function of the difference C₀-C₁ and of the difference C_{H}-C_{B}.

In one or more embodiments, said calculating the quantity of SAP comprises applying a proportionality coefficient z indicative of the molecular weight of the SAP monomeric unit.

In one or more embodiments, said calculating the amount of SAP further comprises applying a correction coefficient A indicative of the crosslinking degree of the SAP.

The sample obtained from post-consumer absorbent sanitary products can be selected from a cellulose sample, a plastic sample, a SAP sample, and mixtures thereof.

The method subject of the present description comprises the step a) wherein a first fraction of the sample in which to measure the quantity of SAP is placed in an aqueous solution containing a known quantity C₀ of a cation X^{Y} to obtain a suspension comprising a solid fraction and a liquid fraction. In step b), after a period of time T₁, the solid fraction is separated from the liquid fraction and the quantity C₁ of residual cation in the liquid fraction is measured after the time T₁. The period of time T₁ of said step a) can range from 2 to 15 hours, preferably 6 hours.

In one or more embodiments, said separating step b) is carried out by filtration of said solid fraction in a collection device. The collected solid fraction can also be washed with an extracting solution.

The Inventors of the present application have taken into consideration that the sample to be quantified, derived from post-consumer absorbent sanitary products, may contain significant traces of the same cation used in step a) of the method, for example, traces of calcium ions, deriving from metabolism. These traces could interfere with the quantitative evaluation, in particular, by causing an underestimation of the free calcium and, therefore, of the SAP content. Consequently, as an alternative to the selection of a water-soluble cation X^{Y} (divalent or trivalent metal) that is not present in the metabolism, the method comprises specific steps that allow obtaining the analytical blank value. In the method in question, as will be exemplified below, the blank value is indicative of a quantity of calcium ions already present in the sample to be tested (according to the difference C_{H} - C_{B}) and which will have determined the precipitation of a part of the SAP present in the sample.

The quantity of SAP is, therefore, calculated as a function of [(C₀- C₁) + (C_{H} -C_{B})].

In one or more embodiments, said proportionality coefficient z and correction coefficient A are applied to the sum of the difference C₀-C₁ and the difference C_{H}-C_{B} or rather, they are applied to [(C₀- C₁) + (C_{H} - C_{B})].

In one or more embodiments, the sample obtained from post-consumer absorbent sanitary products in which to measure the quantity of SAP can be selected from a cellulose sample, a plastic sample, a SAP sample, and mixtures thereof.

In one or more embodiments, said water-soluble cation X^{Y} used and quantified in the different steps of the method is selected from calcium, magnesium, zinc, aluminum, preferably it is the calcium ion (Ca⁺⁺). Preferably, the water-soluble cation X^{Y} used and quantified in the different steps of the method (added or already present in the sample) is the same cation, more preferably it is the calcium ion (Ca⁺⁺).

The cation X^{Y} of the step a) is equal to the cation X^{Y} of the step f), to the cation X^{Y} of the step n), and to the cation X^{Y} of the step o).

In one or more embodiments, said aqueous solution containing a known quantity C₀ of a water-soluble cation may contain a known quantity of Ca⁺⁺ ions in the form of calcium chloride (CaCl₂), calcium nitrate (Ca(NO₃)₂) or calcium sulfate (CaSO₄), preferably granular anhydrous calcium chloride.

The period of time T₂ of said step d) can range from 1 to 3 hours, preferably 2 hours.

The period of time T₃ of said step e) can range from 1 to 3 hours, preferably 2 hours.

In one or more embodiments, the suspension obtained in said step a) and the suspension obtained in said step d) may be kept under stirring for the time period T₁ and for the time period T₂, respectively.

In one or more embodiments, said salt contained in the aqueous solution of step d) may be selected from NaCl, KCl, Na₂SO₄, K₂SO₄, NaNO₃ and KNO₃ preferably NaCl.

In one or more embodiments, the aqueous solution containing an acid has a pH of between 0.5 and 1.5.

In this way, it is possible to obtain a back-titration of the cation, for example, of the free calcium ion, present in a solution deriving from a mass of dry SAP, placed to react in suspension with a solution of calcium ions of known concentration and in adequate excess (at least 5 mmol of ion for each gram of SAP).

The method, therefore, envisages measuring the quantity C₁ of residual cation after the period of time T₁ in the liquid fraction derived from the suspension obtained in step b) and calculating the difference C₀-C₁ between the known quantity C₀ and the quantity C₁ of residual cation after the time T₁.

In one or more embodiments, the quantity of the cation, for example, of calcium ions, may be measured by flame or plasma spectrophotometry, as for example described in EPA Tests Methods 6010D and 7000B. This quantity can be expressed in weight/volume (mg/L).

In particular, by titrating a known quantity of virgin SAP with calcium ions it is possible to determine the mmoles of calcium ions, for example, which are precipitated by the aforesaid quantity, or rather, the quantity of calcium ions subtracted from the solution.

Preferably, the cation X^{Y} used in the method described is the calcium ion used in the form of calcium chloride, nitrate or sulfate. Having to perform a back-titration, a known excess of standard metal is used through the use of concentrated solutions; therefore, it is preferable to select a highly soluble salt. In order for the initial ion concentration used to be as accurate as possible, a salt is used that can be dried to constant weight. CaCl₂·2H₂O, for example, is hygroscopic and melts at drying temperatures; it is therefore preferable to use the salt in anhydrous granular form. In the selection of the salt, the ionic strength, I, is also considered, which is generated by the corresponding cation when the salt is placed in solution, since a high ionic strength may reduce the absorbent capacity of the SAP and, consequently, the reaction times.

In light of the aforesaid considerations, the method envisages the use, preferably, of the calcium ion in the form of granular anhydrous calcium chloride. In any case, regardless of the counter-ion, the method exploits the ability of the cation used, preferably of the calcium ion, divalent metal, to react in an exchange reaction with the sodium, or potassium, or hydrogen ion present in the SAP (and/or in its LPA derivative possibly present in the sample if it had been subjected to oxidative treatments that had involved its de-crosslinking).

The reactions of CLPA and LPA with the cation X^{Y}, for example, the calcium ion lead to the formation of insoluble derivatives, which subtract the calcium from the starting solution, whose analytical concentration C₀ is known. The “missing” calcium may, therefore, be traced back to the total quantity of SAP with the possibility of distinguishing the quantity of CLPA and LPA.

The difference C₀-C₁ indicative of the “missing” calcium subtracted from the link with the SAP can, therefore, be traced back to the total quantity of SAP contained in the sample.

The difference C_{H}-C_{B} is indicative of the quantity of the cation X^{Y} contained in the sample to be quantified, but derived from human metabolism and already bound to the SAP. In the case of calcium, as an example, calcium of metabolic origin may in fact be present in post-consumer absorbent sanitary products in part already bound to the SAP, and in part bound to other negative ions (essentially chlorides and sulfates). These two forms of calcium interfere because they compete with the added calcium, one positively and the other negatively. One has already blocked the SAP, removing it from the reaction with C₀; and the other passes into solution and competes with C₀. Treatment with an aqueous saline solution will allow obtaining a liquid fraction into which the calcium C_{B} passes. Treatment with an acid solution will allow obtaining a liquid fraction into which all the calcium of metabolic origin, C_{H}, passes, including that possibly already bound to the SAP. Therefore, to quantify the correction C_{H}-C_{B}, we proceed on a second fraction of the same sample, first treated with a saline solution and then filtered and treated with an acid solution.

The second fraction of the sample, for example, in a quantity ranging from 1.0 to 10.0 g, to be suspended in the solution comprising a salt, for example, NaCl, is preferably dried. The saline solution comprises a salt that prevents the swelling of the SAP and allows obtaining — in the liquid fraction obtained from the suspension — the passage of free calcium of metabolic origin, C_{B}, free not bound to the SAP present.

Preferably, the solution comprising an acid is an aqueous solution comprising HCl in an amount comprised between 0.1 and 1.0% weight/volume. The filtered solid fraction may be suspended in a volume of acidic aqueous solution equal to 10 times the weight of the sample to be tested. The aqueous acidic solution comprises an acid that prevents the swelling of the SAP and promotes the exchange between the protons H⁺ and the calcium of metabolic origin bound to the SAP, C_{H}, which passes into solution.

Adequate acidity, combined with the ionic strength, in fact, eliminates the absorbent capacity of the SAP and allows the release into solution of any metal excreted but absorbed by the SAP (or by the rest of the matrix, whether it consists of cellulose only, or CLPA or LPA, or their combinations, even with polyolefins). All the calcium that contaminates the post-consumer sanitary products (raw material) would be extracted, determined and considered like this in the quantitative analysis of the SAP, precisely because the fraction of calcium ions of metabolic origin determines the inactivation and precipitation of a portion of the SAP contained in the sample to be quantified.

Measuring the quantity of SAP contained in the sample to be tested is, therefore, calculated as a function of the difference C₀-C₁ and the difference C_{H}-C_{B}.

The quantity in grams of SAP is calculated as {1.22·[C₀–C₁+C_{H}-C_{B}]·94.04} g and expressed in percentage weight as

$$\frac{1.22 \times \left[{C}_{0} - {C}_{1} + {C}_{H} - {C}_{B}\right] \times 94.04}{w e i g h t o f t h e s a m p l e i n g}$$

, %. weight of the sample in g,

In one or more embodiments, the step of calculating the quantity of SAP comprises applying a proportionality coefficient z indicative of the molecular weight of the SAP monomeric unit. This coefficient allows converting the moles of calcium into the mass of SAP equivalent thereto. Choosing as a reference the monomer unit C₃H₃NaO₂ typical of most polyacrylates used in post-consumer absorbent products (PAP), the coefficient z is equal to 94.04 g/mol.

In one or more embodiments, said calculating the amount of SAP further comprises applying a correction coefficient A indicative of the crosslinking degree of the SAP. The expression “degree of crosslinking” means the percentage ratio by mass between the “cross-links” (which do not bind the metal cation but which still contribute to the mass of the SAP) and the monomer units (which bind the aforesaid cation) (Prabhu, et al., International Journal of Pharmaceutical Investigation, 2015, vol. 5 (4), pp. 214-225).

In one or more embodiments, said proportionality coefficient z and correction coefficient A are applied to the sum of the difference C₀-C₁ and the difference C_{H}-C_{B}, or rather, they are applied to [(C₀- C₁) + (C_{H} - C_{B})].

The coefficient A is the value that makes the purity equal to 100% if a sample of known mass of virgin SAP is titrated with a known excess of calcium. It has been hypothesized, and verified, that as calcium ions are added to a suspension of SAP, the concentration of free calcium in solution will be zero until all the anionic sites of the monomer unit to which the cation can bind are saturated, and will then increase linearly, up to the limit of the solubility of the salt used.

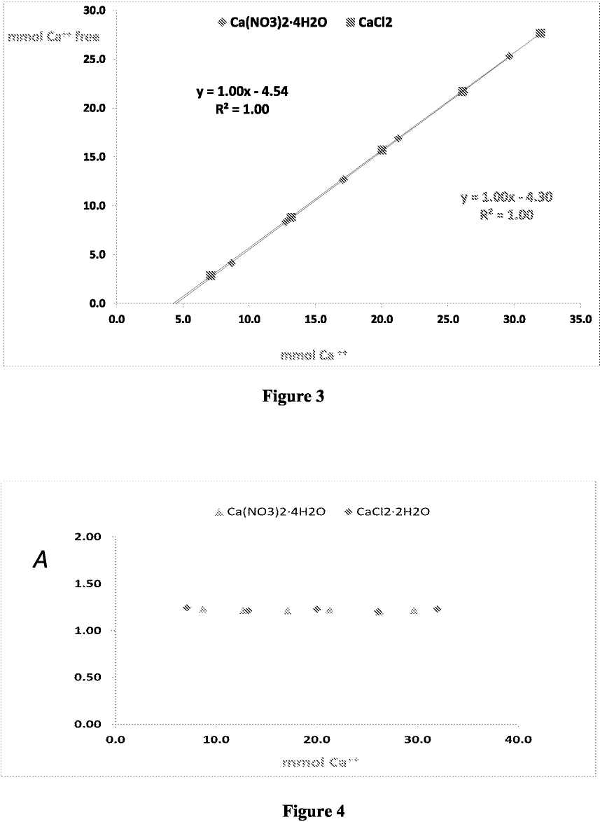

The graph in FIG. 3 reports the relative trend of free calcium as a function of that added at different concentrations to a fixed mass of virgin SAP. The object is to determine the quantity of calcium that the SAP mass can bind, as each Ca⁺⁺ cation exchanges with two of the Na⁺, K⁺ and/or H⁺ present in the SAP. For this purpose, 11 masses of different SAP were taken, all equal to 1.00 g, and suspended in 11 solutions of 500.0 ml of calcium ions at different concentrations. The trend obtained reveals that up to a certain concentration of added calcium, all the calcium remains bound to the insoluble SAP, and there is no free calcium in solution. Once all the COO⁻ sites of the SAP are saturated, the extra calcium added remains in solution and is measured (EPA Tests Methods 6010D or 7000B). The intercept on the x-axis expresses precisely this value equivalent to saturation: each gram of SAP corresponds to (4.4 ± 0.1) mmol of calcium ions.

Once the free moles in divalent calcium ion solution have been determined, and being a pure sample, A will be the factor that makes this relationship true:

$$\frac{2 \times p r e c i p i t a t e d m o l e s \times 94.04}{i n i t i a l m a s s o f S A P} \times \text{A=100\%}$$

If each monomer unit of the LPA bearing a carboxylate weighs 94.04 g/mol, and each divalent calcium ion binds a maximum of two, with a regular LPA polymer chosen as a reference, then the product of twice the moles of precipitated calcium multiplied by the weight of the unit that is repeated in the chain, must correspond to the total weighted mass, except for the coefficient A which depends on the degree of crosslinking.

The coefficient A does not vary in relation to the quantity of calcium ions that can be used (FIG. 4): for the same mass of SAP, the difference between the moles of total calcium added and those of free calcium, measured in solution, remains constant (EPA Tests Methods 6010D or 7000B). This difference coincides with the moles of calcium precipitated because they are bound to the insoluble SAP, and is equal to 4.4 mmol for each gram of SAP. The coefficient A is 1.22 ± 0.02, calculated by making explicit the relation (I) applied to the aforesaid experiments made on pure SAP. The deviation from the unit is attributable to the degree of crosslinking of the SAP, and corrects for the contribution given to the mass of the SAP by the crosslinkers that do not react with the cation.

Consequently, the amount of SAP can be calculated according to the equation E: A · [(C₀-C₁)] · z, wherein A is equal to 1.22 and z is equal to 94.04 g/mol, if we want to express the concentration of SAP as sodium polyacrylate, whose monomer unit is C₃H₃NaO₂ of mass z. The coefficient z equals 108 g/mol if the SAP concentration is expressed as polymethyl acrylate (C₄H₅NaO₂)_{n·}

The midpoint (·) in the equations shown is used to represent multiplication. Considering the possible presence of the cation intrinsically contained in the sample, the amount of SAP can be calculated according to the equation E₁: A · [(C₀ - C₁) + (C_{H}- C_{B})] · z.

The method subject of the present description also allows quantifying the fraction of CLPA and LPA contained in the SAP of the sample to be tested. For this object, the method also comprises the steps of:
i) putting a third fraction of the sample to be subjected to quantification in an aqueous solution containing a salt, preferably NaCl, and obtaining a suspension comprising a solid fraction and a liquid fraction, in order to obtain the solubilization and passage of the LPA fraction of the SAP into liquid phase,
l) obtaining a liquid fraction comprising LPA and a solid fraction comprising CLPA,
m) separating the liquid fraction containing LPA from the solid fraction containing CLPA after a period of time T₄,
n) putting the solid fraction containing CLPA in an aqueous solution containing a known quantity C₀ of said cation X^{Y} for a period of time T₅, and measuring the quantity C₃ of said cation X^{Y} after the time period T₅ in order to obtain the quantity of CLPA in the sample to be tested,
o) adding to the liquid fraction containing LPA a known quantity C₀ of said cation X^{Y} for a period of time T₆, and measuring the quantity C₂ of the cation X^{Y} after the time period T₆ in order to obtain the quantity of LPA in the sample to be tested,
p) calculating the quantity of CLPA as a function of C₀ - C₃ and C_{H} and/or
q) calculating the quantity of LPA as a function of C₀ - C₂ and C_{B}.

In one or more embodiments, the quantity of CLPA can be calculated according to the equation E₂: A · [(C₀ - C₃) + C_{H}] · z and/or said quantity of LPA can be calculated according to the equation E₃: A · [(C₀ - C₂) - C_{B}] · z.

The period of time T₄ of said step m) can range from 2 to 15 hours, preferably 6 hours.

The period of time T₅ of said step n) can range from 1 to 3 hours, preferably 2 hours.

The period of time T₆ of said step o) can range from 1 to 3 hours, preferably 2 hours.

In one or more embodiments, the suspension obtained in said step i) is kept under stirring for the period of time T₄.

In one or more embodiments, said separating step m) is carried out by filtration of said solid fraction in a collection device. The collected solid fraction may also be washed with an extracting solution.

In particular, the method comprises the step i) wherein a third fraction of the sample to be subjected to quantification is suspended in an aqueous solution containing a salt in order to obtain the solubilization and passage of the LPA fraction of the SAP into the liquid phase.

In one or more embodiments, said salt contained in the aqueous solution of step i) may be selected from NaCl, KCl, Na₂SO₄, K₂SO₄, NaNO₃ and KNO₃ preferably NaCl.

In one or more embodiments, the solution comprising a salt comprises NaCl (0.6% by weight).

The aqueous solution containing a salt allows reduction of the swelling of the SAP (as also described in Ohgun, S., Dukjon, K., Theoretical and experimental investigation of the swelling behavior of sodium polyacrylate superabsorbent particles, Journal of Applied Polymers Science , 87, 2, 2003, 252-257; Fan L., et al, Preliminary study of the relationship between water absorbency and zeta potentials of crosslinked poly(acrylic acid), J. Controlled Release, 2011, 152).

This treatment with the saline solution, preferably containing NaCl, favors the solubilization of the LPA fraction of the SAP that will pass into solution (liquid fraction) and the separation of a solid fraction containing the insoluble CLPA fraction of the SAP.

The method comprises a step in which the solid fraction containing CLPA is placed in an aqueous solution containing a known quantity C₀ of the water-soluble cation X^{Y}, preferably calcium ion, for a period of time T₅ and measuring the quantity C₃ of said cation after the time T₅ in order to obtain the quantity of CLPA in the sample to be tested.

The amount of CLPA in the sample to be tested will therefore be related to the difference C₀-C₃ and to C_{H}.

The quantity of CLPA can be calculated according to the equation E₂: A · [(C₀ -C₃) + C_{H}] · z wherein z is indicative of the molecular weight of the monomer unit of the SAP, and the correction coefficient A is indicative of the degree of crosslinking of the SAP. Choosing as a reference the monomer unit C₃H₃NaO₂, typical of most polyacrylates used in post-consumer absorbent products, the coefficient z is equal to 94.04 g/mol. The coefficient A is equal to 1.22.

A known quantity C₀ of said cation X^{Y} is added to the liquid fraction containing the LPA fraction of the SAP for a period of time T₆, and the quantity C₂ of cation is measured after the time T₆.

The amount of LPA in the sample to be tested will, therefore, be related to the sum of the difference C₀ - C₂ and to C_{B}.

Preferably, said first fraction, said second fraction, and said third fraction of the sample derived from post-consumer sanitary products in which to measure the SAP content have the same weight.

The quantity of LPA can be calculated according to the equation E₃: A · [(C₀ - C₂) - C_{B}] · z wherein z is indicative of the molecular weight of the monomer unit of the SAP and the correction coefficient A is indicative of the degree of crosslinking of the SAP. By choosing as a reference for the SAP a crosslinked sodium polyacrylate of monomer unit C₃H₃NaO₂, as a typical component of most polymers used in PAPs, the coefficient z will be equal to 94.04 g mol (108 g/mol if the polymer is based on methyl-acrylate). The coefficient A is equal to 1.22.

The method subject of the present application may also comprise the step of determining the degree of crosslinking (DC) of the pure SAP as a function of the mass contribution of all the crosslinkers present, M_{CL}, and the mass contribution of all the monomeric units present in the SAP, M_{UM}, wherein said degree of crosslinking DC is calculated according to the equation:

$$D C , \% = \frac{{M}_{C L}}{{M}_{U M}} ⋅ 100$$

wherein said whole mass contribution of the monomer units M_{UM} is calculated according to the equation M_{UM}= Y· (C0-C1) · z, wherein the coefficient Y is the charge of the said soluble cation X^{Y}, and z is a proportionality coefficient indicative of the molecular weight of the SAP monomeric unit, and said whole mass contribution of these cross-linkers M_{CL} is calculated according to the equation M_{CL} = M_{Tot} - M_{UM.} The coefficient Y is equal to 2 if calcium ions are used, X^{Y} = Ca²⁺; the proportionality coefficient z is equal to 94.04 g/mol when the SAP chosen as a reference has the monomer unit C₃H₃NaO₂ of mass 94.04 g/mol.

The method, therefore, also allows increasing the degree of crosslinking of the SAP, in which said DC is calculated on a sample of virgin SAP.

In this case, the method for calculating the degree of crosslinking of the SAP in a sample including virgin SAP may include the steps of:
a) contacting the sample of known mass M_{Tot} with an aqueous solution comprising a known quantity C₀ of a water-soluble cation X^{Y} and obtaining a suspension comprising a solid fraction and a liquid fraction,
b) after a period of time T₁, separating the solid fraction from the liquid fraction and measuring the residual quantity C₁ of said water-soluble cation X^{Y} in said liquid fraction,
c) calculating the difference C₀-C₁ between the known quantity C₀ and the quantity C₁ of the cation X^{Y} after the time period T₁ in order to calculate the mass contribution of all the monomer units of the SAP, where said mass contribution M_{UM} is calculated according to the equation M_{UM} = Y· (C₀-C₁) · z, wherein Y is the charge of said water-soluble cation X^{Y}, and z is a proportionality coefficient indicative of the molecular weight of the monomer unit of the SAP,
d) calculating the degree of crosslinking of the SAP as a function of the whole mass contribution of the crosslinkers, M_{CL}, and the whole mass contribution of the monomer units, M_{UM}, of the SAP, wherein said degree of crosslinking DC is calculated according to the equation:

$$D C , \% = \frac{{M}_{C L}}{{M}_{U M}} ⋅ 100$$

wherein said whole mass contribution of the monomer units M_{UM} is calculated according to the equation M_{UM}= Y· (C0- C1) ·z wherein Y is the charge of the said soluble cation X^{Y} and z is a proportionality coefficient indicative of the molecular weight of the SAP monomeric unit, and said whole mass contribution of these cross-linkers M_{CL} is calculated according to the equation M_{CL} = M_{Tot} - M_{UM}, wherein M_{Tot} is the known mass of SAP.

The coefficient Y is equal to 2 if calcium ions are used, X^{Y} = Ca²⁺, where X = Ca and Y = +2; the proportionality coefficient z is equal to 94.04 g/mol when the SAP chosen as a reference has the monomer unit C₃H₃NaO₂.

### EXAMPLES

The following examples describe methods for quantifying the SAP content in a cellulose sample, in a plastic sample (comprising polyolefins, PO), and in a SAP sample. These samples were obtained from a method of treating post-consumer absorbent products, in particular diapers, which included the following steps:
1. autoclave sterilization of post-consumer products, at a temperature between 120° C. and 140° C., and at a pressure between 1 bar and 3.6 bar;
2. shredding the sterilized products using a shredder to obtain sterilized and shredded absorbent sanitary products having a particle size of less than 10 cm, preferably less than 3 cm, more preferably less than 1 cm,
3. drying the shredded products in a dryer in which the drying air temperature inside the dryer 42 is approximately 140° C.,
4. separating the products by at least one centrifugal separator into the different cellulose, plastic and SAP fractions

For the experimental tests described below, each sample of cellulose, plastic, and SAP was divided into three fractions of equivalent weight.

An example of calculating the coefficient A and the degree of crosslinking of the SAP is also described.

### Analytical Method for Determining the Quantity of SAP as Sodium Polyacrylate Contained in Solid Matrices Comprising Cellulose

A laboratory sample (about 20 g) was homogenized by size reduction of the particle size (according to the UNI 10802: 2013 Method) to fragments of between 2 and 3 cm.

The sample was dried at a temperature of 110° C. and was divided into three fractions (or aliquots) of 5.00 g.

### 1.1 Measurement of the Total Amount of SAP (Comprising CLPA and LPA)

A first aliquot (first fraction) of the sample was used to measure the total amount of SAP, comprising CLPA and LPA.

A quantity equal to 1.00 g of calcium chloride was dissolved in 500 ml of water under stirring. Once the molecular weight of the salt is known, the moles of total calcium C₀ are calculated, dividing 1.00 g by 111 g/mol, therefore 9.01 mmol.

The aforesaid solution comprising calcium chloride was poured onto a first fraction of the sample to be tested (5.00 g) placed in a 1 L beaker. The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers, carried out by washing with an extracting solution (150, 200 and 250 ml of deionized water) for 10 minutes each, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The quantity of moles of non-precipitated free calcium ions, or C₁, evaluated by atomic absorption spectrophotometry with flame atomization, was found to be 3.82 mmol (EPA test method 7000B).

The quantity expressed as moles/L of calcium precipitated by the SAP is equal to the difference between the quantity of calcium ions C₀ and the quantity of calcium ions C₁ after the time T₁, or rather, the difference C₀ - C₁, therefore 5.19 mmol per liter of solution.

At the same time, 3.0 g of NaCl was dissolved in 500 ml and poured onto the second 5.00 g fraction of the same sample to be tested, placed in a 1 L beaker, under stirring. The saline solution prevents the swelling of the SAP and allows a release solution to be obtained, into which free calcium of metabolic origin, C_{B}, which is not bound to the SAP present, also passes. The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on NaCl, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The quantity of moles of non-precipitated free calcium ions, or C_{B}, evaluated by atomic absorption spectrophotometry with flame atomization, was found to be 0.22 mmol compared to the unit volume.

The solid is recovered from the filter, and suspended in a 1 L beaker in the presence of HCl (250 ml) at pH=1 for 1 h. The acid solution prevents the swelling of the SAP and promotes the exchange between the protons H⁺ and the calcium of metabolic origin bound to the SAP, C_{H}, which passes into the releasing solution. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on HCl, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The quantity of moles of free calcium ions C_{H}, evaluated by atomic absorption spectrophotometry with flame atomization, was found to be 0.83 mmol compared to the unit volume.

The quantity expressed as moles/L of calcium derived from metabolism and indicative of only calcium bound to the SAP is equal to the difference between the quantity of calcium ions C_{H} and the quantity of calcium ions C_{B} after the time T₁, or rather, the difference C_{H} - C_{B}, found to be equal to 0.61 mmol.

The total amount of SAP, calculated according to the equation A · [(C₀ - C₁) + (C_{H} - C_{B})]· 94.04 g/mol in which A is equal to 1.22, was equal to 0.665 g, corresponding to 13.3% by weight of the initial sample of 5.00 g.

### 1.2 Measuring the Quantity of the Individual CLPA and LPA Fractions of the SAP

The third 5.00 g fraction of the dried sample to be subjected to quantification was placed in a solution comprising 3.0 g of NaCl dissolved in 500 ml of water inside a 1 L beaker. The saline solution prevents the swelling of the SAP and allows a release solution to be obtained, into which the fraction of the soluble SAP, or rather, the LPA, also passes. The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on NaCl, and filtering in the same Buchner funnel. The filtered solution was brought to 1.00 dm³ with deionized water (part of the extracting solvent used in the transfers remains absorbed by the matrix), and supplemented with 1.00 g of calcium chloride (Part A). The solid recovered entirely from the filter is instead transferred into a 1 L beaker containing 500 ml of water with 1.00 g of dissolved CaCl₂ (Part B).

### Part A. Determination of the Soluble Fraction Based on LPA

The filtered solution, brought to 1.00 dm³ with deionized water and supplemented with 1.00 g of calcium chloride (C₀ = 9.01 mmol) was kept under stirring for 6 hours to allow the precipitation of the insoluble calcium salt deriving from the crosslinking action operated by the divalent ion on the sodium or potassium LPA. After 6 hours, 50.0 ml of solution was collected, centrifuged for 15 minutes at 4000 rpm, and filtered with 0.45 µm filters. The quantity of moles of non-precipitated free calcium ions, or C₂, evaluated by atomic absorption spectrophotometry with flame atomization, was found to be 8.2 mmol compared to the unit volume. Since it was 0.22 mmol for the same sample C_{B}, it follows that for 5.00 g of sample analyzed, the weight percentage of LPA in the initial sample is 1.4%, based on the equation E₃: A · [(C₀ - C₂) - C_{B})] · 94.04 g/mol wherein A is equal to 1.22.

### Part. B Determination of the Insoluble Fraction CLPA

The solid recovered entirely from the filter was transferred into a 1L beaker containing 500 ml of water with 1.00 g of dissolved CaCl₂ (C₀= 9.01 mmol). The suspension is kept under stirring for 6 hours to allow the precipitation of the calcium salt deriving from the sodium or potassium CLPA, following the exchange reaction of the divalent ion with the monovalent one. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on NaCl, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The quantity of moles of non-precipitated free calcium ions, or C₃, evaluated by atomic absorption spectrophotometry with flame atomization, was found to be 5.02 mmol compared to the unit volume. Since it was 0.83 mmol for the same sample C_{H}, it follows that for 5.00 g of sample analyzed, the weight percentage corresponding to the CLPA contained in the initial sample is 11.1%, based on the equation E₂: A . [(C₀ - C₃) + C_{H}] · 94.04 g/mol wherein A is equal to 1.22. The value of 11.1% is consistent within one percentage point with the value of 11.9%, obtainable by difference between what was obtained in example 1.1 (13.3%) and that in Part A of this same example (1.4%).

### Analytical Method for Determining the Quantity of SAP as Alkaline Polyacrylate Contained in Solid Matrices Comprising Plastic

A laboratory sample (about 50 g) was homogenized by size reduction of the particle size (according to the UNI 10802: 2013 Method) to fragments of between 2 and 3 cm. The sample was dried at a temperature of 110° C. and was divided into three aliquots of 10.00 g.

### 2.1 Measurement of the Total Amount of SAP (Comprising CLPA and LPA)

A first aliquot (first fraction) of the sample was used to measure the total amount of SAP, comprising CLPA and LPA.

A quantity equal to 1.00 g of calcium chloride was dissolved in 500 ml of water under stirring. Once the molecular weight of the salt is known, the moles of total calcium C₀ are calculated, dividing 1.00 g by 111 g/mol, therefore 9.01 mmol.

The aforesaid solution comprising calcium chloride was poured onto a first fraction of the sample to be tested (10.00 g) placed in a 1 L beaker. The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers, carried out by washing with an extracting solution (150, 200 and 250 ml of deionized water) for 10 minutes each, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The quantity of moles of non-precipitated free calcium ions, or rather C₁, evaluated by means of atomic absorption spectrophotometry with flame atomization, was equal to 6.87 mmol.

The quantity expressed as moles/L of calcium precipitated by the SAP is equal to the difference between the quantity of calcium ions C₀ and the quantity of calcium ions C₁ after the time T₁ or the difference C₀ - C₁, therefore 2.14 mmol per liter of solution.

At the same time, 3.0 g of NaCl was dissolved in 500 ml and poured onto the second 10.00 g fraction of the same sample to be tested, placed in a 1 L beaker, under stirring. The saline solution prevents the swelling of the SAP and allows a release solution to be obtained, into which free calcium of metabolic origin, C_{B}, which is not bound to the SAP present, also passes. The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on NaCl, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered again with 0.45 µm filters. The quantity of moles of non-precipitated free calcium ions, or rather, C_{B}, evaluated by atomic absorption spectrophotometry with flame atomization, was found to be 0.05 mmol, compared to the unit volume.

The solid is recovered from the filter, and suspended in a 1 L beaker in the presence of HCl (250 ml) at pH=1 for 1 h. The acid solution prevents the swelling of the SAP and promotes the exchange between the protons H⁺ and the calcium of metabolic origin bound to the SAP, C_{H}, which passes into the releasing solution. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on HCl, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The quantity of moles of free calcium ions C_{H}, evaluated by means of atomic absorption spectrophotometry with flame atomization, resulted equal to 0.24 mmol, compared to the unit volume.

The quantity expressed as moles/L of calcium derived from metabolism and indicative of only calcium bound to the SAP is equal to the difference between the quantity of calcium ions C_{H} and the quantity of calcium ions C_{B} after the time T₁, or rather, the difference C_{H} - C_{B}, found to be equal to 0.19 mmol.

The total amount of SAP, calculated according to the equation A · [(C₀ - C₁) + (C_{H} - C_{B})]· 94.04 g/mol in which A is equal to 1.22, was equal to 0.267 g, corresponding to 2.7% by weight of the initial sample of 10.00 g.

### 2.2 Measuring the Quantity of the Individual CLPA and LPA Fractions of the SAP

The third 10.00 g fraction of the dried sample to be subjected to quantification was placed in a solution comprising 3.0 g of NaCl dissolved in 500 ml of water inside a 1 L beaker. The saline solution prevents the swelling of the SAP and allows a release solution to be obtained, into which the fraction of the soluble SAP, or rather, the LPA, also passes. The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on NaCl, and filtering in the same Buchner funnel. The filtered solution was brought to 1.00 dm³ with deionized water (part of the extracting solvent used in the transfers remains absorbed by the matrix), and supplemented with 1.00 g of calcium chloride (Part A). The solid recovered entirely from the filter is instead transferred into a 1 L beaker containing 500 ml of water with 1.00 g of dissolved CaCl₂ (Part B).

### Part A. Determination of the Soluble Fraction Based on LPA

The filtered solution, brought to 1.00 dm³ with deionized water and supplemented with 1.00 g of calcium chloride (C₀ = 9.01 mmol) is kept under stirring for 6 hours to allow the precipitation of the insoluble calcium salt deriving from the crosslinking action operated by the divalent ion on the sodium or potassium LPA. After 6 hours 50.0 ml of solution were taken, centrifuged for 15 minutes at 4000 rpm, and filtered with 0.45 µm filters. The quantity of moles of non-precipitated free calcium ions, or C₂, evaluated by atomic absorption spectrophotometry with flame atomization, was found to be 8.92 mmol compared to the unit volume. Since it was 0.05 mmol for the same sample C_{B}, it follows that for 10.00 g of sample analyzed, the weight percentage of LPA in the initial sample is 0.05%, based on the equation E₃: A . [(C₀ - C₂) - C_{B})]· 94.04 g/mol wherein A is equal to 1.22.

### Part. B Determination of the Insoluble Fraction CLPA

The solid recovered entirely from the filter is transferred into a 1 L beaker containing 500 ml of water with 1.00 g of dissolved CaCl₂ (C₀= 9.01 mmol). The suspension is kept under stirring for 6 hours to allow the precipitation of the calcium salt deriving from the sodium or potassium CLPA, following the exchange reaction of the divalent ion with the monovalent one. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on NaCl, and filtering in the same Buchner funnel. The filtered solution was brought with deionized water to 1.00 dm³ (part of the extracting solvent used in the transfers remains absorbed by the matrix), and a quantity equal to 50 ml was taken, centrifuged for 15 minutes at 4000 rpm, and filtered again with 0.45 µm filters. The quantity of moles of non-precipitated free calcium ions, or C₃, evaluated by atomic absorption spectrophotometry with flame atomization, was found to be 7.00 mmol compared to the unit volume. Since it was 0.24 mmol for the same sample C_{H}, it follows that for 10.00 g of sample analyzed, the weight percentage corresponding to the CLPA contained in the initial sample is 2.6 %, based on the equation E₂: A . [(C₀ - C₃) + C_{H}] · 94.04 g/mol wherein A is equal to 1.22. The value of 2.6% is consistent with the value of 2.65%, obtainable by difference between that obtained in example 2.1 (2.7%) and that in Part A of this same example (0.05%).

### Analytical Method for Determining the Quantity of SAP as Alkaline Polyacrylate Contained in Solid Matrices Comprising SAP

A laboratory sample (about 5 g) was homogenized by size reduction of the particle size (according to the UNI 10802: 2013 Method) to fragments of between 2 and 3 cm.

The sample was dried at a temperature of 110° C. and was divided into three aliquots of 1.00 g.

### 3.1 Measuring of the Total Quantity of SAP (Comprising CLPA and LPA)

A first aliquot (first fraction) of the sample was used to measure the total amount of SAP, comprising CLPA and LPA.

A quantity equal to 5.00 g of calcium chloride was dissolved in 500 ml of water under stirring. Once the molecular weight of the salt is known, the moles of total calcium C₀ are calculated, dividing 5.00 g by 111 g/mol, therefore 45.04 mmol.

The aforesaid solution comprising calcium chloride was poured onto a first fraction of the sample to be tested (1.00 g) placed in a 1 L beaker. The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers, carried out by washing with an extracting solution (150, 200 and 250 ml of deionized water) for 10 minutes each, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The quantity of moles of non-precipitated free calcium ions, or rather C₁, evaluated by means of atomic absorption spectrophotometry with flame atomization, was equal to 39.06 mmol.

The quantity expressed as moles/L of calcium precipitated by the SAP is equal to the difference between the quantity of calcium ions C₀ and the quantity of calcium ions C₁ after the time T₁, or rather, the difference C₀ - C₁, therefore 6.34 mmol per liter of solution.

At the same time 3.0 g of NaCl was dissolved in 500 ml and poured onto the second 1.00 g fraction of the same sample to be tested, placed in a 1 L beaker, under stirring. The saline solution prevents the swelling of the SAP and allows a release solution to be obtained, into which free calcium of metabolic origin, C_{B}, which is not bound to the SAP present, also passes. The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on NaCl, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The quantity of moles of non-precipitated free calcium ions, or rather C_{B}, evaluated by atomic absorption spectrophotometry with flame atomization, was equal to 0.20 mmol, compared to the unit volume.

The solid is recovered from the filter, and suspended in a 1 L beaker in the presence of HCl (250 ml) at pH=1 for 1 h. The acid solution prevents the swelling of the SAP and promotes the exchange between the protons H⁺ and the calcium of metabolic origin bound to the SAP, C_{H}, which passes into the releasing solution. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on HCl, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The amount of moles of free calcium ions C_{H}, evaluated by atomic absorption spectrophotometry with flame atomization, resulted equal to 0.55 mmol, compared to the unit volume.

The quantity expressed as moles/L of calcium derived from metabolism and indicative of only calcium bound to the SAP is equal to the difference between the quantity of calcium ions C_{H} and the quantity of calcium ions C_{B} after the time T₁, or rather, the difference C_{H} - C_{B}, found to be equal to 0.35 mmol.

The total amount of SAP, calculated according to the equation A · [(C₀ - C₁) + (C_{H} - C_{B})]· 94.04 g/mol in which A is equal to 1.22, was equal to 0.727 g, corresponding to 72.7% by weight of the initial sample of 1.00 g.

### 3.2 Measuring the Quantity of the Individual CLPA and LPA Fractions of the SAP

The third 1.00 g fraction of the dried sample to be subjected to quantification was placed in a solution comprising 3.0 g of NaCl dissolved in 500 ml of water inside a 1 L beaker. The saline solution prevents the swelling of the SAP and allows a release solution to be obtained, into which the fraction of the soluble SAP, or rather, the LPA, also passes. The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on NaCl, and filtering in the same Buchner funnel. The filtered solution was brought with deionized water to 1.00 dm³ (part of the extracting solvent used in the transfers remains absorbed by the matrix), and supplemented with 5.00 g calcium chloride (Part A). The solid recovered entirely from the filter is instead transferred into a 1 L beaker containing 500 ml of water with 5.00 g of dissolved CaCl₂ (Part B).

### Part A. Determination of the SOLUBLE Fraction Based on LPA

The filtered solution, brought to 1.00 dm³ with deionized water and supplemented with 5.00 g of calcium chloride (C₀ = 45.04 mmol) is kept under stirring for 6 hours to allow the precipitation of the insoluble calcium salt deriving from the crosslinking action operated by the divalent ion on the sodium or potassium LPA. After 6 hours 50.0 ml of solution were taken, centrifuged for 15 minutes at 4000 rpm, and filtered with 0.45 µm filters. The quantity of moles of non-precipitated free calcium ions, or C₂, evaluated by atomic absorption spectrophotometry with flame atomization, was found to be 44.78 mmol compared to the unit volume. Since it was 0.20 mmol for the same sample C_{B}, it follows that for 1.00 g of sample analyzed, the weight percentage of LPA in the initial sample is 0.07%, based on the equation E₃: A . [(C₀ - C₂) - C_{B})]· 94.04 g/mol wherein A is equal to 1.22.

### Part. B Determination of the Insoluble Fraction CLPA

The solid recovered entirely from the filter is transferred into a 1 L beaker containing 500 ml of water with 5.00 g of dissolved CaCl₂ (C₀= 45.04 mmol). The suspension is kept under stirring for 6 hours to allow the precipitation of the calcium salt deriving from the sodium or potassium CLPA, following the exchange reaction of the divalent ion with the monovalent one. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers of 10 minutes each, carried out by successive washes with 150, 200 and 250 ml of the same extraction solution based on NaCl, and filtering in the same Buchner funnel. The filtered solution was brought with deionized water to 1.00 dm³ (part of the extracting solvent used in the transfers remains absorbed by the matrix), and a quantity equal to 50 ml was taken, centrifuged for 15 minutes at 4000 rpm, and filtered again with 0.45 µm filters. The quantity of moles of non-precipitated free calcium ions, or C₃, evaluated by atomic absorption spectrophotometry with flame atomization, was found to be 39.55 mmol compared to the unit volume. Since it was 0.55 mmol for the same sample C_{H}, it follows that for 1.00 g of sample analyzed, the weight percentage corresponding to the CLPA contained in the initial sample is 69.3 %, based on the equation E₂: A ·[(C₀ - C₃) + C_{H}] · 94.04 g/mol wherein A is equal to 1.22. The value of 69.3% is consistent within 2.7 percentage points with the value of 72%, obtainable by difference between what was obtained in example 3.1 (72.7%) and that in Part A of this same example (0.7%).

As is evident from the examples above, the reactions of CLPA and LPA with calcium ion lead to the formation of insoluble derivatives, which subtract calcium from the starting solution, whose analytical concentration C₀ is known. The “missing” calcium may, therefore, be traced back to the total quantity of SAP with the possibility of distinguishing the quantity of CLPA and LPA.

### Analytical Method for Determining the Crosslinking Coefficient A and The Degree of Crosslinking DC

A sample of 5 g pure SAP based on sodium polyacrylate was dried at a temperature of 110° C. to constant weight. Finally, it was divided into three aliquots of 1.00 g in order to obtain an average value over three measures of A and of DC

A quantity equal to 2.90 g of calcium chloride was dissolved in 500 ml of water under stirring. Once the molecular weight of the salt is known, the moles of total calcium C₀ are calculated, dividing 2.9 g by 111 g/mol, therefore 26.13 mmol.

The first fraction of the sample to be tested was added to the aforesaid solution comprising calcium chloride (M_{Tot}= 1.00 g). The suspension was stirred rapidly with a glass rod and stirring was maintained for 6 hours. After the aforesaid period, the solid matrix was filtered into a collection device, or rather, a Buchner funnel with 1 mm and 300 ml holes, in turn placed on a 1.0 dm³ cylinder. The solid matrix was recovered from the filter and subjected to three successive transfers, carried out by washing with an extracting solution (150, 200 and 250 ml of deionized water) for 10 minutes each, and filtering in the same Buchner funnel. The filtered solution was brought to 1.00 dm³ with deionized water (part of the extracting solvent used in the transfers remains absorbed by the matrix), and a quantity equal to 50 ml was taken, centrifuged for 15 minutes at 4000 rpm, and filtered further with 0.45 µm filters. The quantity of moles of non-precipitated free calcium ions, or rather, C₁, evaluated by atomic absorption spectrophotometry with flame atomization, was equal to 21.77 mmol.

The quantity expressed as moles/L of calcium precipitated by the SAP is equal to the difference between the quantity of calcium ions C₀ and the quantity of calcium ions C₁ after the time T₁ or the difference C₀ - C₁, therefore 4.36 mmol per liter of solution. If a crosslinked sodium polyacrylate-based reference sample is chosen, its corresponding mass which has precipitated divalent calcium ions will correspond to M_{UM} = Y· (C₀-C₁) ·z, wherein Y is the charge of said calcium cation, soluble in water. Therefore being X^{Y} = Ca²⁺, Y = +2. The proportionality coefficient z indicative of the molecular weight of the monomer unit of the SAP is equal to 94.04 g/mol, if a cross-linked sodium polyacrylate with a monomer unit C₃H₃NaO₂ of mass 94.04 g/mol is chosen as the reference sample for the SAP. Therefore M_{UM} will be equal to 2· 4.36 mmol · 94.04 g/mol, or rather M_{UM}= 0.820 g. By contrast, the whole mass contribution of the crosslinkers M_{CL} is calculated according to the equation M_{CL} = M_{Tot} - M_{UM} = (1.00 - 0.820) g equal to 0.180 g, being M_{Tot}= 1.00 g.

In relation to this test which gives 4.36 mmol, having used a pure reference sample of 1.00 g, the coefficient A will instead be the value that makes the relation 0.820 g · A = 1.00 g true, therefore A= 1.22.

Similarly to what was done for the first 1.00 g fraction, in order to obtain an average value of A, the second and third fractions of the sample chosen as reference were balanced, respectively, with a quantity equal to 2.22 g of calcium (20.00 mmol) and with one equal to 1.47 g (13.23 mmol) in two distinct beakers. After 6 hours of reaction and subsequent filtrations and reconstitutions of the 1 liter solutions, the quantities of moles of non-precipitated free calcium ions, evaluated by atomic absorption spectrophotometry with flame atomization, were, respectively, equal to 15.71 mmol if 1.00 g of pure SAP was equilibrated with 2.22 g of calcium (20.00 mmol), and 8.80 mmol, if 1.00 g of pure SAP was equilibrated with 1.47 g of calcium (13.23 mmol). The quantity expressed as moles/L of calcium precipitated by the SAP is therefore, respectively, 4.29 mmol and 4.43 mmol.

In relation to the test that gives 4.29 mmol, M_{UM} = Y· (C₀-C₁) ·z, (wherein Y is the charge of the said calcium cation, X^{Y} = Ca²⁺, therefore Y=2, and the proportionality coefficient z, indicative of the molecular weight of the monomer unit of the SAP, is 94.04 g/mol), corresponds to 2 · 4.29 mmol · 94.04 g/mol. Therefore, a M_{UM} is obtained equal to 0.807 g. Consequently, the whole mass contribution of the crosslinkers M_{CL} will be given by the equation M_{CL} = M_{Tot} - M_{UM} = (1.00 - 0.807) g equal to 0.193 g, being M_{Tot}= 1.00 g. The coefficient A will instead be the value that makes the relation 0.807 g · A = 1.00 g true, therefore A equals 1.24.

In relation to the test that gives 4.43 mmol, M_{UM} = Y· (C₀-C₁) ·z, (wherein Y is the charge of the said soluble cation, XY = Ca^{2+,} therefore Y=2, and the proportionality coefficient z, indicative of the molecular weight of the monomer unit of the SAP, is 94.04 g/mol), corresponds to 2 · 94.04 g/mol · 4.43 mmol. Therefore, a M_{UM} is obtained that is equal to 0.833 g, and a M_{CL} = M_{Tot} - M_{UM} = (1.00 - 0.833) g equal to 0.167 g, being M_{Tot}= 1.00 g. The coefficient A will instead be the value that makes the relation 0.833 g · A = 1.00 g true, therefore A equals 1.20.

This results, in the face of three measurements, in an average value of the coefficient A equal to 1.22 ± 0.02.

The “degree of crosslinking”, understood as the percentage ratio in mass between the contribution of all the crosslinkers, M_{CL}, and that of all the remaining monomer units, M_{UM}, will also be determinable with the same data set, according to the equation:

$$D C , \% = \frac{{M}_{C L}}{{M}_{U M}} ⋅ 100$$

where said whole mass contribution of monomer units M_{UM} is calculated according to the equation M_{UM} = Y· (C₀-C₁) ·z, wherein Y is the charge of the said soluble calcium cation Ca²⁺, X^{Y} = Ca²⁺, therefore Y= 2, and z is the proportionality coefficient indicative of the molecular weight of the monomer unit of the SAP, equal to 94.04 g/mol when the pure SAP chosen as reference has the monomer unit of C₃H₃NaO₂. Said mass contribution of these crosslinkers M_{CL} is instead calculated according to the equation M_{CL} = M_{Tot} -M_{UM}, wherein M_{Tot} is the known mass of weighed SAP, equal to 1.00 g in the example.

In terms of weight, for each gram of cross-linked sodium polyacrylate, chosen as reference for the SAP, therefore M_{Tot}= 1.00 g, the mass contribution M_{UM} of the non-cross-linked part based on polyacrylate that binds calcium is equal — in the aforesaid three separate tests — to 0.820 g, 0.807 g, 0.833 g, as calculated above. This results in an average value of M_{UM} = (0.820 ± 0.013) g. By difference for each gram of SAP, for the contribution M_{CL} of the cross-linkers, the calculated values of 0.180 g, 0.193 g, 0.167 g result, from which an average value of M_{CL}= (0.180 ± 0.013) g arises. The equation DC, % =

$$D C , \% = \frac{{M}_{C L}}{{M}_{U M}} ⋅ 100$$

therefore allows an average value to be obtained on three DC measurements equal to (22.0 ± 1.3)%.

The method subject of the present description therefore makes it possible to: i) measure the total quantity of super absorbent polymers (SAP) in a sample obtained from post-consumer absorbent sanitary products (previously subjected to at least one treatment that comprises the separation of the portions of SAP, cellulose and plastic) and ii) to measure the quantity of the CLPA fraction and the LPA fraction contained in the SAP.

### Method for Directly Measuring the Quantity of Linear Polyacrylate (LPA) Polymers and/or Cross-Linked Polyacrylate (CLPA) Polymers

The method subject of this description may also allow direct quantification of the CLPA fraction and the LPA fraction contained in the SAP of the sample to be tested, as described in the previous sections, and optionally subsequently the total quantity of SAP. In this case, the method is a method for measuring the quantity of super absorbent polymers (SAP) in a sample obtained from post-consumer absorbent sanitary products comprising at least one portion of a portion of cellulose and/or a portion of plastic in addition to a portion of SAP, said post-consumer absorbent sanitary products having been, preferably, previously subjected to at least one treatment comprising the separation of said portions, wherein the SAP contained in said sample comprise polymers of linear polyacrylate (LPA) and/or polymers of cross-linked polyacrylate (CLPA), the method comprising the steps of:
a) contacting a first fraction of the sample with a solution comprising a salt for a period of time T₂ and obtaining a suspension comprising a solid fraction and a liquid fraction, wherein said salt promotes the passage of said water-soluble cation X^{Y} of metabolic origin and not already bound to the SAP into said liquid fraction,
b) separating the solid fraction from the liquid fraction and contacting, for a period of time T₃, the solid fraction with a solution comprising an acid for displacing said water-soluble cation X^{Y} of metabolic origin already bound to the SAP and promoting its passage into said solution;
c) measuring the quantity C_{B} of said water-soluble cation X^{Y} contained in said liquid fraction obtained in step a) after the period of time T₂, and the quantity C_{H} of said water-soluble cation X^{Y} contained in said aqueous solution containing an acid, after the time period T₃,
d) contacting a second fraction of the sample with an aqueous solution containing a salt and obtaining a suspension comprising a solid fraction and a liquid fraction in order to obtain the solubilization and the passage of the LPA fraction of the SAP into liquid phase,
e) obtaining a liquid fraction comprising LPA and a solid fraction comprising CLPA,
f) separating the liquid fraction containing LPA from the solid fraction containing CLPA after a period of time T₄,
g) contacting the solid fraction containing CLPA with an aqueous solution containing a known quantity C₀ of said water-soluble cation X^{Y} for a period of time T₅, and measuring the quantity C₃ of said cation X^{Y} after the time period T₅ in order to obtain the quantity of CLPA in the sample to be tested,
h) adding to the liquid fraction containing LPA a known quantity C₀ of said water-soluble cation X^{Y} for a period of time T₆ and measuring the quantity C₂ of the cation X^{Y} after the time period T₆ in order to obtain the quantity of LPA in the sample to be tested,
i) calculating the quantity of CLPA as a function of C₀ - C₃ and C_{H} and/or
l) calculating the quantity of LPA as a function of C₀ - C₂ and C_{B}.

The period of time T₂ and the period of time T₃ can range from 1 to 3 hours, preferably 2 hours.

The period of time T₄ can range from 2 to 15 hours, preferably 6 hours. The period of time T₅ can range from 1 to 3 hours, preferably 2 hours. The period of time T₆ can range from 1 to 3 hours, preferably 2 hours.

In one or more embodiments, said separating step b) is carried out by filtration of said solid fraction in a collection device. The collected solid fraction may also be washed with an extracting solution.

In particular, the method comprises the step d) wherein a second fraction of the sample to be subjected to quantification is suspended in an aqueous solution containing a salt in order to obtain the solubilization and passage of the LPA fraction of the SAP into the liquid phase.

In one or more embodiments, said salt contained in the aqueous solution of step d) may be selected from NaCl, KCl, Na₂SO₄, K₂SO₄, NaNO₃ and KNO₃ preferably NaCl. In one or more embodiments, the solution comprising a salt comprises NaCl (0.6% by weight).

Calculating the quantity of the SAP (CLPA e LPA) comprises applying the proportionality coefficient z indicative of the molecular weight of the SAP monomeric unit.

Calculating the quantity of SAP (CLPA and LPA) also comprises applying the correction coefficient A indicative of the degree of crosslinking (DC) of the SAP.

The coefficient A is equal to 1.22 and the proportionality coefficient z is equal to 94.04 g/mol when the SAP chosen as a reference has the monomer unit C₃H₃NaO₂.

According to the method described, the quantity of CLPA is calculated according to the equation E₂: A · [(C₀ - C₃) + C_{H}] · z and/or the quantity of LPA is calculated according to the equation E₃: A · [(C₀ - C₂) - C_{B}] · z.

The water-soluble cation X^{Y} is selected from calcium, magnesium, zinc, aluminum, preferably calcium ion (Ca⁺⁺).

The sample obtained from post-consumer absorbent sanitary products is selected from a cellulose sample, a plastic sample, a SAP sample, and mixtures thereof.

The method also comprises the step of determining the degree of crosslinking DC of the SAP as a function of the ratio between the whole mass of the crosslinkers, M_{CL}, and the whole mass of the monomer units of the SAP, M_{UM}, wherein said degree of crosslinking DC is calculated according to the equation:

$$D C , \% = \frac{{M}_{C L}}{{M}_{U M}} ⋅ 100$$

wherein said whole mass contribution of the monomer units M_{UM} is calculated according to the equation M_{UM} = Y· (C₀ - C₁) · z wherein Y is the soluble cation charge and z is said proportionality coefficient indicative of the molecular weight of the SAP monomeric unit, and said mass contribution of these cross-linkers M_{CL} is calculated according to the equation, M_{CL} = M_{Tot} - M_{UM} wherein Mₜₒₜ is the weight of said sample comprising SAP.

The coefficient z is equal to 94.04 g/mol when the monomeric unit of the SAP reference is C₃H₃NaO₂, and said coefficient Y is equal to 2 when said water-soluble cation X^{Y} is selected from calcium, magnesium and zinc.

Of course, without prejudice to the principle of the invention, the details of construction and the embodiments may be widely varied, without thereby departing from the scope of the invention as defined by the claims that follow.

1. A method for measuring the quantity of super absorbent polymers (SAP) in a sample obtained from post-consumer absorbent sanitary products comprising at least one portion of a portion of cellulose and/or a portion of plastic in addition to a portion of SAP, said post-consumer absorbent sanitary products having been, preferably, previously subjected to at least one treatment comprising the separation of said portions,
wherein the SAP contained in said sample comprise polymers of linear polyacrylate (LPA) and/or polymers of cross-linked polyacrylate (CLPA), the method comprising the steps of:
a) contacting a first fraction of the sample with an aqueous solution containing a known quantity C₀ of a water-soluble cation X^{Y}, and obtaining a suspension comprising a solid fraction and a liquid fraction,
b) after a period of time T₁, separating the solid fraction from the liquid fraction and measuring the residual quantity C₁ of said water-soluble cation X^{Y} in said liquid fraction,
c) calculating the difference C₀-C₁ between the known quantity C₀ and the quantity C₁ of the cation X^{Y} remaining after the time period T₁,
d) contacting a second fraction of the sample with a solution comprising a salt for a period of time T₂ and obtaining a suspension comprising a solid fraction and a liquid fraction, wherein said salt promotes the passage of said water-soluble cation X^{Y} of metabolic origin and not already bound to the SAP into said liquid fraction,
e) separating the solid fraction from the liquid fraction and contacting, for a period of time T₃, the solid fraction with a solution comprising an acid for displacing said water-soluble cation X^{Y} of metabolic origin already bound to the SAP and promoting its passage into said solution,
f) measuring the quantity C_{B} of said water-soluble cation X^{Y} contained in said liquid fraction obtained in step d) after the period of time T₂, and the quantity C_{H} of said water-soluble cation X^{Y} contained in said aqueous solution containing an acid, preferably HC1, after the time period T³,
g) calculating the difference C_{H} - C_{B} this difference being indicative of the quantity of said cation contained in the sample and derived from the metabolism and bound to the SAP,
h) calculating the quantity of SAP contained in said sample as a function of the difference C₀-C₁ and of the difference C_{H}-C_{B}.

2. The method according to claim 1, wherein said calculating the quantity of SAP comprises applying a proportionality coefficient z indicative of the molecular weight of the SAP monomeric unit.

3. The method according to claim 2, wherein said calculating the quantity of SAP also comprises applying a correction coefficient A indicative of the degree of crosslinking of the SAP.

4. The method according to claim 3, wherein said coefficient A is equal to 1.22 and wherein said proportionality coefficient z is equal to 94.04 g/mol when the monomeric unit of the SAP reference is C₃H₃NaO₂.

5. The method according to claim 3, wherein said quantity of SAP is calculated according to the equation E₁: A · [(C₀ - C₁) + (C_{H} - C_{B})] · z.

6. The method according to claim 3, wherein the method further comprises the steps:
i) contacting a third fraction of the sample with an aqueous solution containing a salt, preferably NaCl, and obtaining a suspension comprising a solid fraction and a liquid fraction in order to obtain the solubilization and the passage of the LPA fraction of the SAP into liquid phase,
l) obtaining a liquid fraction comprising LPA and a solid fraction comprising CLPA,
m) separating the liquid fraction containing LPA from the solid fraction containing CLPA after a period of time T₄,
n) contacting the solid fraction containing CLPA with an aqueous solution containing a known quantity C₀ of said cation X^{Y} for a period of time T₅ and measuring the quantity C₃ of said cation X^{Y} after the time period T₅ in order to obtain the quantity of CLPA in the sample to be tested,
o) adding to the liquid fraction containing LPA a known quantity C₀ of said water-soluble cation X^{Y} for a period of time T₆ and measuring the quantity C₂ of the cation X^{Y} after the time period T₆ in order to obtain the quantity of LPA in the sample to be tested,
p) calculating the quantity of CLPA as a function of C₀ - C₃ and C_{H} and/or
q) calculating the quantity of LPA as a function of C₀ - C₂ and C_{B}.

7. The method according to claim 6, wherein said quantity of CLPA is calculated according to the equation E₂: A · [(C₀ - C₃) + C_{H}] · z and/or said quantity of LPA is calculated according to the equation E₃: A · [(C₀ - C₂) - C_{B}] · z.

8. The method according to claim 1, wherein said water-soluble cation X^{Y} is selected from calcium, magnesium, zinc, aluminum, preferably calcium ion (Ca⁺⁺).

9. The method according to claim 1, wherein said sample obtained from post-consumer absorbent sanitary products is selected from a cellulose sample, a plastic sample, a sample of SAP and mixtures thereof.

10. The method according to claim 1, wherein the salt contained in the solution of step d) is selected from the group consisting of NaCl, KCl, Na₂SO₄, K₂SO₄, NaNO₃ and KNO₃, preferably NaCl.

11. The method according to claim 2, wherein the method also comprises a step for determining the degree of crosslinking — DC — of the SAP, as a ratio of the whole mass of cross-linkers, M_{CL}, to the whole mass of the SAP monomers, M_{UM},
wherein said degree of crosslinking DC is calculated according to the equation:
$$D C , \% = \frac{{M}_{C L}}{{M}_{U M}} ⋅ 100$$
wherein said whole mass contribution of the monomers M_{UM} is calculated according to the equation M_{UM} = Y· (C₀ - C₁) ·z wherein Y is the soluble cation charge and z is said proportionality coefficient indicative of the molecular weight of the SAP monomeric unit, and said whole mass contribution of cross-linkers M_{CL} is calculated according to the equation: M_{CL} = M_{Tot} - M_{UM}, wherein Mₜₒₜ is the weight of said sample comprising SAP.

12. The method according to claim 11, wherein said coefficient z is equal to 94.04 g/mol when the monomeric unit of the SAP reference is C₃H₃NaO₂, and said coefficient Y is equal to 2 when said water-soluble cation X^{Y} is selected from calcium, magnesium and zinc.
